(19) United States
(12) Patent Application Publication
(10) Pub. No.: US 2015/0014211 A1
(43) Pub. Date: Jan. 15, 2015

(54) Medical Kits

(71) Applicant: ASCLEMED USA, INC. dba EnovaChem Manufacturing, Torrance, CA (US)

(72) Inventor: Robert Prentiss Nickell, Torrance, CA (US)

(21) Appl. No.: 14/332,275

(22) Filed: Jul. 15, 2014

Related U.S. Application Data
(60) Provisional application No. 61/846,541, filed on Jul. 15, 2013.
(60) Provisional application No. 61/914,958, filed on Dec. 12, 2013.
(60) Provisional application No. 61/925,123, filed on Jan. 8, 2014.
(60) Provisional application No. 61/935,796, filed on Feb. 4, 2014.
(60) Provisional application No. 61/978,968, filed on Apr. 13, 2014.
(60) Provisional application No. 61/984,625, filed on Apr. 25, 2014.
(60) Provisional application No. 62/015,348, filed on Jun. 20, 2014.

(51) Int. Cl. A61B 19/02; A61J 1/16

(52) U.S. Cl. CPC A61B 19/02; A61J 1/16; A61B 2019/0202
USPC 206/572; 206/363; 206/438; 206/570

(57) ABSTRACT

Provided is a medical kit with all the necessary components for a medical practitioner in the office practice setting to inject a variety of anesthetics. The kits can comprise a holder, such as a box, and active pharmaceutical ingredients (API), with each API placed in a separate container in a form that is ready for injection, the API selected from the group consisting of: a. lidocaine hydrochloride and triamcinolone acetonide with or without a separate container of ammonia; b. bupivacaine hydrochloride (HCl) and lidocaine hydrochloride (HCl), and optionally one or more of triamcinolone acetonide, methylprednisolone acetate, or dexamethasone sodium; c. bupivacaine hydrochloride (HCl) and one or more of triamcinolone acetonide or methylprednisolone acetate; d. Methylprednnisolone acetate and lidocaine hydrochloride (HCl), and optionally bupivacaine hydrochloride (HCl); e. methylprednisolone acetate and lidocaine hydrochloride (HCl), and bupivacaine hydrochloride (HCl); f. lidocaine hydrochloride, triamcinolone acetonide, and ammonia; g. bupivacaine hydrochloride or lidocaine HCl, and betamethasone sodium phosphate and betamethasone acetate; h. bupivacaine hydrochloride or lidocaine HCl, and dexamethasone sodium phosphate; i. ketorolac tromethamine, lidocaine HCl, and optionally bupivacaine hydrochloride; and j. one or more of dexamethasone sodium.

### CROSS-REFERENCE

The present application claims the benefit of provisional U.S. Appl. No. 61/846,541 dated Jul. 15, 2013, of provisional U.S. Appl. No. 61/914,958, filed on Dec. 12, 2013, provisional U.S. Appl. No. 61/925,123, filed on Jan. 8, 2014, provisional U.S. Appl. No. 61/935,796, filed on Feb. 4, 2014, provisional U.S. Appl. No. 61/978,968, filed on Apr. 13, 2014, provisional U.S. Appl. No. 61/984,625, filed on Apr. 25, 2014, provisional U.S. Appl. No. 62/015,348, filed on Jun. 20, 2014, all of which are incorporated herein by reference in their entirety.

### BACKGROUND SECTION OF THE INVENTION

With the changing reimbursements, types of health care insurance, and new regulations with regards to treatment, a medical practitioner and his/her team when treating a patient is under immense time pressures to perform quickly, efficiently and with safety for the patient. If a patient requires a singular or even multiple injections, the medical practitioner must rely on his/her team to first locate the necessary and correct injectable medications, insure they are the correct strength and percentage, insure dating is current, insure the top has not been contaminated, insure the liquid is not crystallized, or containing growth, insure there is sufficient medication in the vial, and insure that the injection has been stored at the proper temperature and humidity as well as all other possible related materials and safety precautions needed to treat the patient. The medical practitioner can lose valuable time trying to not only locate the necessary drugs and materials to treat the patient but as well to insure the compliance, the safety of the patient by preventing contamination. In some instances, not all the drugs and materials may be readily available, in the current exam room, resulting in delayed treatment and possible and potential medical errors. In addition, the medical practitioner or his/her team may make a mistake by incorrectly choosing the drug, by incorrectly identifying the product, not checking the expiration date, not returning it to the proper environment for storage, or not having enough product and thus requiring an additional sterile vial to be opened to inject directly into the veins of a patient.

There is a need in the art for a convenience kit that solves the above mentioned problems in the medical practitioner office.

### SUMMARY SECTION OF THE INVENTION

In one embodiment, provided is a kit comprising the following active pharmaceutical ingredients (API), with each ingredient placed in a separate container in a form that is ready for injection, wherein the API is selected from the group consisting of:
a. lidocaine hydrochloride and triamcinolone acetonide with no separate container of ammonia;
b. bupivacaine hydrochloride (HCl) and lidocaine hydrochloride (HCl), and optionally one or more of triamcinolone acetonide, methylprednisolone acetate, or Dexamethasone Sodium;
c. bupivacaine hydrochloride (HCl) and one or more of triamcinolone acetonide or methylprednisolone acetate;
d. Methylprednnisolone acetate and lidocaine hydrochloride (HCl), and optionally bupivacaine hydrochloride (HCl);
e. Methylprednisolone acetate and lidocaine hydrochloride (HCl), and bupivacaine hydrochloride (HCl);
f. lidocaine hydrochloride, triamcinolone acetonide, and ammonia;
g. (Bupivacaine hydrochloride or lidocaine HCl), and Betamethasone Sodium Phosphate and Betamethasone acetate;
h. (Bupivacaine hydrochloride or lidocaine HCl), and Dexamethasone Sodium phosphate; and
i. Ketorolac Tromethamine, lidocaine HCl, and optionally Bupivacaine hydrochloride
j. one or more of Dexamethasone Sodium.

Each of the above ingredients can be placed in a holder that is preferably a box.

The kit can further comprise povidone-iodine swab stick or pad. The kit can further comprise an antibiotic. The kit can further comprise one or more gloves. The kit can further comprise a mask. The kit can further comprise a bandage. The kit can further comprise gauze. The kit can further comprise alcohol swabs or pads. The kit can further comprise povidone-iodine swabsticks or pads. The kit can further comprise a container of sterile water. The kit can further comprise a container of saline water. The kit can comprise sterile nitrile gloves. The kit can further comprise a sterile drape with a hole or fenestrations. The kit can further comprise a CSR wrap. The kit can comprise an instruction sheet. The kit can comprise a drape. The above listed components can be placed in a package or a container that can be opened by a medical practitioner. The kit can include any 2, any 3, any 4, or any 5 of these components.

The holder can be a box. A tray can be placed inside the box. The kit can comprise three or more of the following: antibiotic, gloves, mask, bandage, gauze, alcohol swabs, sterile water, saline water, a sterile drape, CSR wrap, and instruction sheet. The kit can comprise lidocaine hydrochloride and triamcinolone acetonide with or without a separate container of ammonia. The kit can comprise bupivacaine hydrochloride (HCl) and lidocaine hydrochloride (HCl), and optionally one or more of triamcinolone acetonide, methylprednisolone acetate, or dexamethasone Sodium. The kit can comprise bupivacaine hydrochloride (HCl) and one or more of triamcinolone acetonide or methylprednisolone acetate. The kit can comprise methylprednisolone acetate and lidocaine hydrochloride (HC), and optionally bupivacaine hydrochloride (HCl). The kit can comprise methylprednisolone acetate and lidocaine hydrochloride (HCl), and bupivacaine hydrochloride (HCl). The kit can comprise lidocaine hydrochloride, triamcinolone acetonide, and ammonia. The kit can comprise bupivacaine hydrochloride or lidocaine HCl, and betamethasone sodium phosphate and betamethasone acetate. The kit can comprise bupivacaine hydrochloride or lidocaine HCl, and dexamethasone sodium phosphate. The kit can comprise ketorolac tromethamine, lidocaine HCl, and optionally bupivacaine hydrochloride. The kit can comprise one or more of dexamethasone Sodium.

The kit can comprise: a) One container of bupivacaine hydrochloride; b) Two containers of triamcinolone acetonide; c) One or more packages of povidone-iodine swabsticks; d) gloves; e) bandage; and f) gauze. The kit can comprise: a) One container of bupivacaine hydrochloride; b) One container of lidocaine HCl; c) One or more povidone-iodine swabsticks; d) gloves; e) bandage; and g) gauze. The kit can comprise: a) One container of bupivacaine hydrochloride; b) One container of lidocaine HCl; c) Two containers of triamcinolone acetonide; d) One or more povidone-iodine swabsticks; e) gloves; f) bandage; and g) gauze. The kit can comprise: a) One container of bupivacaine hydrochloride; b) Two containers of lidocaine HCl; c) Three containers of triamcinolone acetonide; d) One or more povidone-iodine swabsticks; e) gloves; f) bandage; and g) gauze. The kit can comprise: a) One container of bupivacaine hydrochloride; b) Two containers of lidocaine HCl; c) Two containers of triamcinolone acetonide; d) One or more povidone-iodine swabsticks; e) gloves; f) bandage; and g) gauze. The kit can comprise: a) Two containers of lidocaine HCl; b) One container of triamcinolone acetonide; c) One or more povidone-iodine swabsticks; d) gloves; e) bandage; and f) gauze. The kit can comprise: a) one container of methylprednisolone acetate; b) two containers of lidocaine HCl; c) one or more of alcohol swabs; d) gauze; e) gloves; and f) bandage. The kit can comprise: a) two containers of triamcinolone acetonide; b) one container of lidocaine HCl; c) one or more containers of ammonia inhalant; d) one or more of povidone-iodine prep pads; e) gloves; and f) CSR wrap. The kit can compromise: a) one container of triamcinolone acetonide; b) one container of lidocaine HCl; c) one or more container of ammonia inhalant ampule; d) one or more of povidone-iodine prep pads; e) a package of gloves; and f) CSR wrap. The kit can comprise: a) one container of ketorolac tromethamine; b) one container of lidocaine HCl; c) one or more of povidone-iodine swabsticks; d) gloves; e) bandage; and f) gauze. The kit can comprise: a) one container of ketorolac tromethamine; b) one container of lidocaine HCl; c) one container of bupivacaine hydrochloride; d) one or more of povidone-iodine swabsticks; e) gloves; f) bandage; and g) gauze. The kit of claim 1, wherein the kit comprises: a) one container of lidocaine HCl; b) one container of bupivacaine hydrochloride; c) one or more of povidone-iodine swabsticks; d) gloves; e) gauze; and f) bandage. The kit can comprise: a) one container of betamethasone; b) one container of lidocaine HCl; c) one or more of povidone-iodine swabsticks; d) gloves; and e) gauze. The betamethasone can be in form of betamethasone sodium phosphate and betamethasone acetate. The kit can comprise: a) one container of dexamethasone sodium phosphate; b) one container of lidocaine HCl; c) one or more povidone-iodine swabsticks; d) gloves; and e) gauze. The kit can comprise: a) one container of ketorolac tromethamine; b) one container of lidocaine HCl; c) one container of bupivacaine hydrochloride; d) one or more povidone-iodine Swabsticks; e) gloves; f) bandage; and g) gauze. The kit can comprise: a) one container of betamethasone; b) one or more containers of bupivacaine hydrochloride; c) one or more povidone-iodine swabsticks; d) gloves; and e) gauze. The betamethasone can be in form of betamethasone Sodium phosphate and betamethasone acetate. The kit can comprise: a) one container of dexamethasone sodium phosphate; b) one container of bupivacaine hydrochloride, c) one or more povidone-iodine swabsticks; d) gloves; and e) gauze. The kit can comprise: a) one container of methylprednisolone acetate; b) one container of bupivacaine hydrochloride; c) one container of lidocaine HCl; d) one container of saline; e) one container of water; f) one or more povidone-iodine swabsticks; g) one or more alcohol pads; h) gloves; and i) drape. The methylprednisolone acetate can be present in 40 mg or 80 mg. The kit can comprise: a) One container of bupivacaine hydrochloride; b) One container of lidocaine HCl; c) One or more povidone-iodine swabsticks; d) gloves; e) bandage; and f) gauze. The kit can comprise: a) One container of dexamethasone sodium phosphate; b) One container of lidocaine HCl; c) One container of bupivacaine hydrochloride, d) one or more povidone-iodine swabsticks; e) gloves; and f) gauze. The kit can comprise: a) One container of methylprednisolone acetate; b) One container of bupivacaine hydrochloride; c) one or more povidone-iodine Prep pads; d) gloves; and e) CSR wrap. The kit can comprise: a) one container of bupivacaine hydrochloride; b) one container of lidocaine HCl; c) two containers of triamcinolone acetonide; d) one or more povidone-iodine swabsticks; e) gloves; f) bandage; and g) gauze. The kit can comprise: a) Two containers of dexamethasone sodium phosphate; b) One or more povidone-iodine swabsticks; c) Gloves; and d) Gauze.

The kit can comprise: a. one container of bupivacaine hydrochloride (10 ml); and b. two containers of triamcinolone acetonide (1 ml). The kit can comprise: a. one container of bupivacaine hydrochloride (10 ml) and b. one container of lidocaine HCl 2% (2 ml). The kit can comprise: a. one container of bupivacaine hydrochloride (10 ml); b. one container of lidocaine HCl (2 ml); and c. two containers of triamcinolone acetonide (1 ml). The kit can comprise: a. one container of bupivacaine hydrochloride (10 ml); b. two containers of lidocaine HCl (2 ml); and c. three containers of triamcinolone acetonide (l ml). The kit can comprise: a. one container of bupivacaine hydrochloride (10 ml); b. two containers of Lidocaine HCl (2 ml); and c. two containers of Triamcinolone acetonide (1 ml). The kit can comprise: a. two containers of Lidocaine HCl (2 ml); and b. one container of Triamcinolone acetonide. The kit can comprise one container of methylprednisolone acetate and one container of lidocaine HCl. The kit can comprise one container of methylprednisolone acetate and two containers of lidocaine HCl. The kit can comprise two containers of Methylprednisolone acetate and two containers of lidocaine HCl. The kit can comprise one container of Triamcinolone acetonide and one container of lidocaine HCl. The kit can comprise one container of Methylprednisolone acetate Injection (Depo-Medrol®) and one container of Lidocaine HCl. The kit can comprise one container of Methylprednisolone acetate Injection (Depo-Medrol®) and two containers of Lidocaine HCl. The kit can comprise two containers of Methylprednisolone acetate Injection (Depo-Medrol®) and two containers of Lidocaine HCl. The kit can comprise one container of Triamcinolone acetonide and one container of Lidocaine HCl. The kit can comprise one container of Lidocaine HCl, one container of bupivacaine hydrochloride, and one container of Methylprednisolone acetate (Depo-Medrol®) (40 mg/mL or 80 mg/mL). The kit can comprise one container of ketorolac and one container of lidocaine HCl. The kit can comprise one container of ketorolac, one container of lidocaine HCl, and one container of bupivacaine hydrochloride. The kit can comprise one container of lidocaine HCl, and one container of bupivacaine hydrochloride.

The kit can comprise: a. one container of bupivacaine hydrochloride (10 ml); b. two containers of triamcinolone acetonide (1 ml); c. a package containing povidone-iodine swab sticks; d. a pair of sterile nitrile gloves; e. bandage; and f. at least about 10 ply gauze. The kit can comprise: a. one container of bupivacaine hydrochloride (10 ml); b. one container of lidocaine HCl 2% (2 ml); c. a package of povidone-iodine swab sticks; d. a pair of sterile nitrile gloves; e. bandage; and f. at least about 10 ply gauze. The kit can comprise: a. one container of bupivacaine hydrochloride (10 ml); b. one container of lidocaine HCl 2% (2 ml); c. two containers of triamcinolone acetonide (1 ml); d. a package of povidone-iodine swab sticks; e. a pair of sterile nitrile gloves; f. bandage; and g. at least about 10 ply gauze. The kit can comprise a. one container of bupivacaine hydrochloride (10 ml); b. two containers of lidocaine HCl 2% (2 ml); c. three containers of triamcinolone acetonide (1 ml); d. a package of povidone-iodine swab sticks; e. a pair of sterile nitrile gloves; f. adhesive bandage; and g. at least about 10 ply gauze. The kit can comprise: a. one container of bupivacaine hydrochloride (10 ml); b. two containers of lidocaine HCl 2% (2 ml); c. two containers of triamcinolone acetonide (1 ml); d. a package of povidone-iodine swab sticks swabs; e. a pair of sterile nitrile gloves; f. bandage; and g. at least about 10 ply gauze. The kit can comprise: a. two containers of lidocaine HCl 2% (2 ml); b. one container of triamcinolone acetonide (1 ml); c. a package of povidone-iodine swab sticks; d. a pair of sterile nitrile gloves; e. adhesive bandage; and f. at least about 10 ply gauze.

The kit can comprise Methylprednisolone (Depo-Medrol®), lidocaine, optionally bupivacaine hydrochloride, alcohol swabs (pads), optionally Povidone-Iodine Swabsticks, gauze, sterile nitrile gloves, bandage, and optionally a sterile drape with a hole. The kit can comprise one container of methylprednisolone 40 mg/ml (1-5 mL) (Depo-Medrol®), one containers of lidocaine HCl 1% (10-20 mL), one container of bupivacaine hydrochloride 0.5% (10 mL), about 6 (or another quantity) packages of alcohol swabs (pads), sterile nitrile gloves, bandage, and a drape with a hole. The kit can comprise one container of methylprednisolone 40 mg/ml (5 mL) (Depo-Medrol®), two containers of Lidocaine 1% (2 mL), about 6 (or another quantity) packages of alcohol swabs (pads), gauze, sterile nitrile gloves, and bandage. The kit can comprise two containers of methylprednisolone 40 mg/ml (1 mL) (Depo-Medrol®), two containers of Lidocaine 1% (2 mL), about 6 (or another quantity) packages of alcohol swabs (pads), gauze, sterile nitrile gloves, and bandage. The kit can comprise one container of Triamcinolone acetonide (1 ml), one container of Lidocaine 1% (10 ml), one container of Ammonia inhalant ampule, one Povidone-iodine prep pad, a pair of Sterile Latex Gloves (size 7.5), and one Sterile drape (w/hole).

The kit can comprise one container of lidocaine HCl (5 ml), one container of bupivacaine hydrochloride (0.25%) (10 ml), one container of Methylprednisolone acetate (Depo-Medrol®) 40 or 80 mg/mL, and one or more of containers of sterile water, saline, sterile drape with a hole, povidone-iodine swabsticks, sterile gloves, and alcohol swabs (pads).

) The kit can comprise two or three of the following active ingredients: one container of Ketorolac 30 mg/ml (1 ml), one container of lidocaine HCl (1% or 2%), one container of Marcaine (0.25%) (10 ml), and one or more of Povidone-Iodine Swabsticks, sterile gloves, bandage, gauze, and spot adhesive bandage.

The kit can comprises a box containing a tray, the tray having 5 cavities, with 4 cavities in parallel fashion to each other, and a fifth cavity abutting all the four cavities and having an area that is substantially same or bigger than combined area of the four cavities.

### BRIEF DESCRIPTION OF THE DRAWINGS

FIG. 1 illustrates a box where a plurality of components are placed therein.

FIG. 2 illustrates a tray with multiple cavities that is placed inside the box.

FIG. 3 illustrates a box in which an ampoule or a vial can be placed.

FIG. 4 illustrates a vial.

FIG. 5 illustrates an ampoule.

FIG. 6 illustrates a swab stick and packaging.

FIG. 7 illustrates a glove.

FIG. 8 illustrates a package and a gauze.

FIG. 9 illustrates a package and a spot bandage.

FIG. 10 illustrates a package and a strip bandage.

FIG. 11 illustrates multi-ply gauze.

FIG. 12 illustrates a mask.

FIG. 13 illustrated an instruction sheet.

FIG. 14 illustrates a CSR wrap.

FIG. 15 illustrated a drape with an opening.

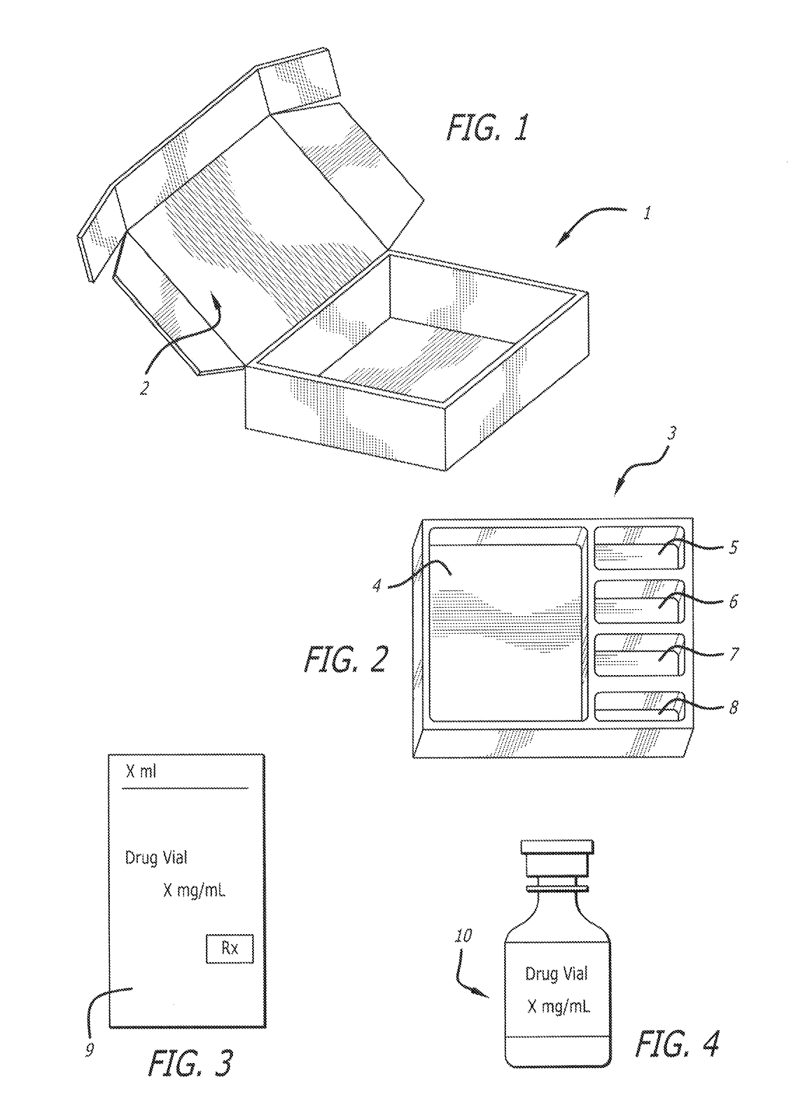

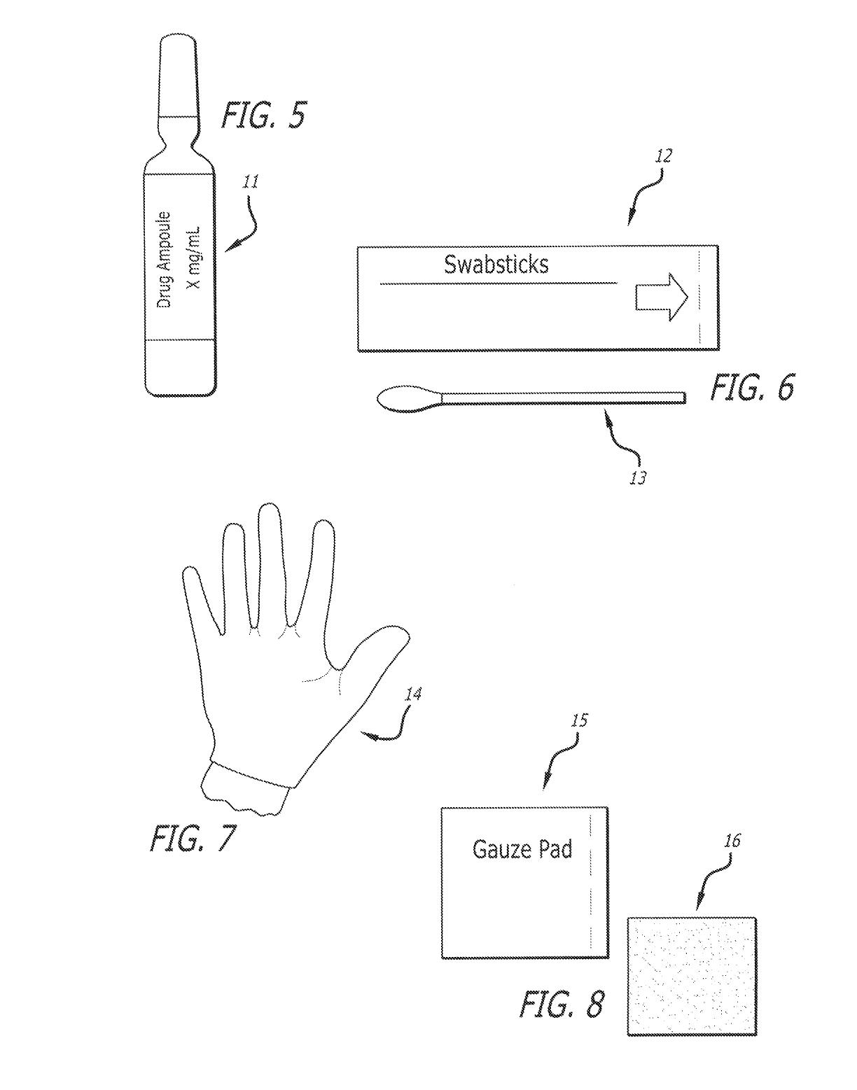

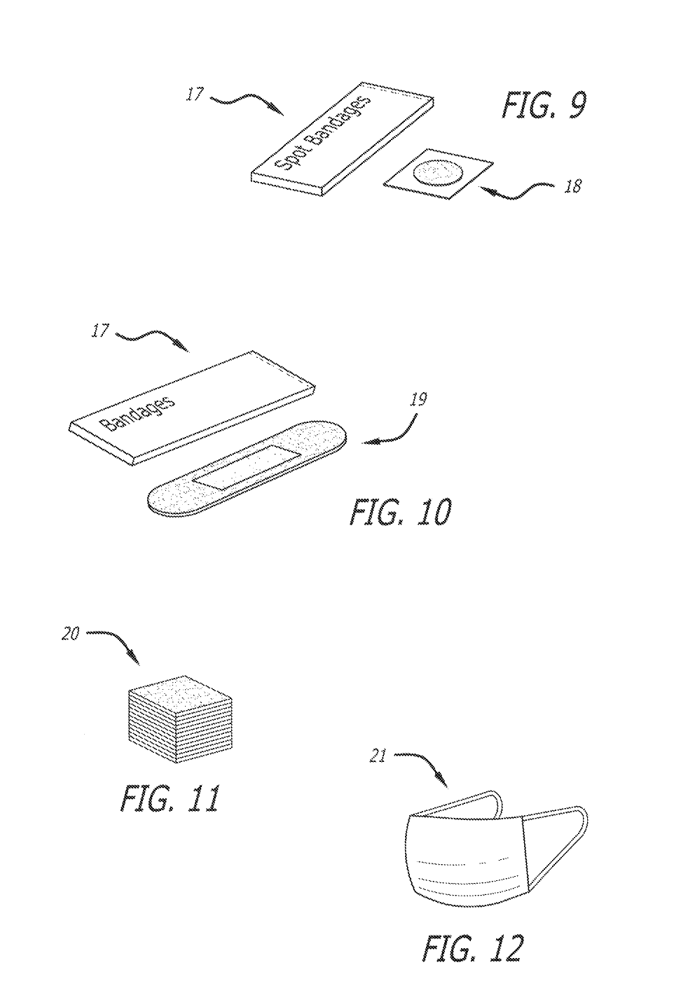

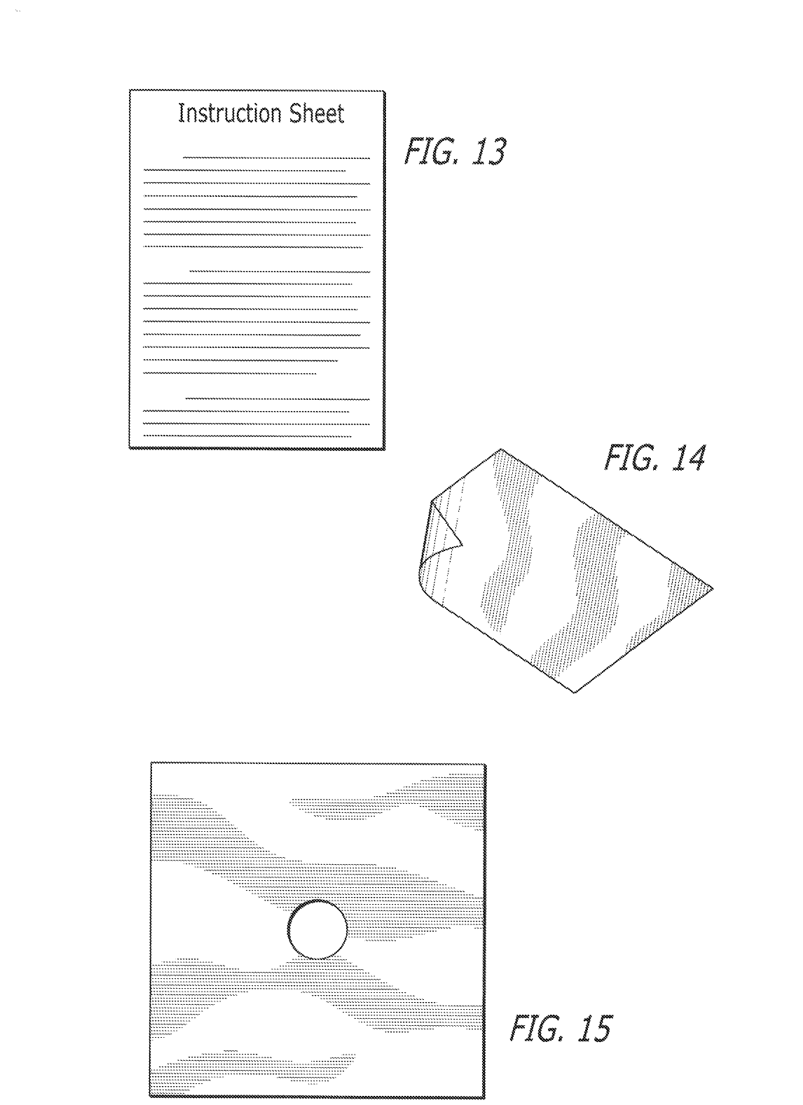

### DETAILED DESCRIPTION OF THE INVENTION

The present invention provides a kit with all the necessary components for a medical practitioner in the office practice setting to inject two or more of a variety of anesthetics including bupivacaine hydrochloride (HCl), or lidocaine hydrochloride (HCl), as well as a combination of corticosteroids including triamcinolone acetonide or methylprednisolone to a patient. Additional active pharmaceutical ingredients include Betamethasone Sodium Phosphate and Betamethasone acetate, and Dexamethasone Sodium Phosphate. The kit can comprise or consist essentially of two or three of these and other active pharmaceutical ingredients plus the medical devices, and medical supplies needed for the particular patient at that particular time of treatment. The active pharmaceutical ingredients can be in a single dose or a multiple dose container.

One component of the medical kit can be bupivacaine hydrochloride (Marcaine®). The chemical name for bupivacaine hydrochloride is 2-Piperidinecarboxamide, 1-butyl-N-(2,6-dimethylphenyl)-hydrochloride. Marcaine® can be a sterile isotonic solution containing sodium chloride. The pH of these solutions can be adjusted to between 4 and 6.5 with sodium hydroxide or hydrochloric acid. A container (ampoule (11) or vial (10)) of bupivacaine hydrochloride in a form (such as a suspension or solution) that is ready for injection comes with the kit, thus eliminating the possible confusing of selecting the incorrect medication. Preferably a container of Marcaine® (USP 10 ml) is used, but any amount from 1 ml to 15 ml could be placed in the kit depending on the type of injection required for the particular treatment. All a medical practitioner has to do is draw fluid with a syringe from the container and administer the drug to a patient by an injection without the worry of selecting the wrong product, having it be out of date or not containing enough medication. The bupivacaine hydrochloride can be administered directly to the joint, vein, or muscle tissue by injection. The instruction sheet for Marcaine® (USP 10 ml) is incorporated herein by reference.

One component of the medical kit can be lidocaine HCl (Xylocaine®), which has the chemical name acetamide, 2-(diethylamino)-N-(2,6-dimethylphenyl)-, monohydrochloride. Xylocaine® is a sterile isotonic solution containing sodium chloride. Xylocaine® can have its pH adjusted with NaOH or HCl. A container (ampoule (11) or vial (10) of lidocaine hydrochloride in a form (such as a suspension or solution) that is ready for injection is placed in the kit. All a medical practitioner has to do is draw fluid with a syringe from the container and administer the drug to a patient by an injection. The lidocaine HCl can be administered parenterally by injection. The instruction sheet for Xylocaine® is incorporated herein by reference.

One component of the medical kit can be triamcinolone acetonide injectable suspension (Kenalog®-40 Injection). Each mL of the sterile aqueous suspension of Kenalog®-40 provides 40 mg triamcinolone acetonide, with sodium chloride for isotonicity, 0.99% (w/v) benzyl alcohol as a preservative, 0.75% carboxymethylcellulose sodium, and 0.04% polysorbate 80 in an aqueous suspension. Sodium hydroxide or hydrochloric acid may have been added to adjust pH between 5.0 and 7.5. Triamcinolone acetonide is a synthetic glucocorticoid corticosteroid with marked anti-inflammatory action. Triamcinolone acetonide can be a sterile aqueous suspension suitable for intralesional and intra-articular injection. The chemical name of triamcinolone acetonide is 9-Fluoro-11β,16α,17,21-tetrahydroxy-pregna-1,4-diene-3,20-dione cyclic 16,17-acetal with acetone. A container (ampoule (11) or vial (10)) of triamcinolone acetonide in a form (such as a suspension or solution) that is ready for injection is a component of the kit. The triamcinolone acetonide can be administered intra-muscular or intra-articular by injection. The instruction sheet for Kenalog®-40 is incorporated herein by reference.

One component of the kit can be methylprednnisolone, preferably methylprednisolone acetate (Depo-Medrol®). The chemical name for methylprednisolone acetate is pregna-1,4-diene-3,20-dione, 21(acetyloxy)-11,17-dihydroxy-6-methyl-,(6α, 11β). Depo-Medrol® is an anti-inflammatory glucocorticoid for intramuscular, intra-articular, soft tissue or intralesional injection. It is available in three strengths: 20 mg/mL; 40 mg/mL; 80 mg/mL of Methylprednisolone acetate. Each milliliter (mL) of these preparations contains Methylprednisolone acetate, polyethylene glycol, polysorbate, monobasic sodium phosphate, Dibasic sodium phosphate, Benzyl alcohol added as a preservative. Sodium chloride can be added to adjust tonicity. When necessary, pH was adjusted with sodium hydroxide and/or hydrochloric acid. A container (ampoule (11) or vial (10)) of methylprednisolone acetate in a form (such as a suspension or solution) that is ready for injection is a component of the kit. The instruction sheet for Depo-Medrol® is incorporated herein by reference.

One component of the kit can be ketorolac or ketorolac tromethamine (Toradol®), which is a non-steroidal anti-inflammatory drug (NSAID). Ketorolac is indicated for short-term management of moderate to severe pain. Toradol® is provided as a solution for injection. The chemical name for ketorolac is (±)-5-benzoyl-2,3-dihydro-1H-pyrrolizine-1-carboxylic acid, 2-amino-2-(hydroxymethyl)-1,3-propanediol. A container (ampoule (11) or vial (10)) of ketorolac or ketorolac tromethamine in a form (such as a suspension or solution) that is ready for injection is a component of the kit. The instruction sheet for Toradol® is incorporated herein by reference.

One components of the kit can be Betamethasone Sodium Phosphate, Betamethasone Acetate, Betamethasone, and mixtures of two or more of them. Betamethasone is a corticosteroid. Betamethasone sodium phosphate chemically is 9-Fluoro-11β,17,21-trihydroxy-16β-methylpregna-1,4-diene-3,20-dione 21-(disodium phosphate). Betamethasone acetate is 9-Fluoro-11β,17,21-trihydroxy-16β-methylpregna-1,4-diene-3,20-dione 21-acetate. Betamethasone sodium phosphate and betamethasone acetate injectable suspension, USP can be a sterile aqueous suspension containing betamethasone as betamethasone sodium phosphate and betamethasone acetate. Inactive ingredients can include dibasic sodium phosphate, monobasic sodium phosphate, edetate disodium, and benzalkonium chloride. The pH can be adjusted to between 6.8 and 7.2. A container (ampoule (11) or vial (10)) in a form (such as a suspension or solution) that is ready for injection can be a component of the kit.

One components of the kit can be dexamethasone sodium phosphate (APP Pharmaceuticals, LLC). Dexamethasone sodium phosphate is a synthetic adrenocortical steroid with a chemical name of 9-fluoro-11β,17-dihydroxy-16α-methyl-21-(phosphonooxy)pregna-1,4-diene-3,20-dione disodium salt. Dexamethasone sodium phosphate injection can be a sterile solution for intravenous, intramuscular, intra-articular, intralesional and soft tissue administration. Each mL of the injection can contain the following components: dexamethasone sodium phosphate, sodium citrate, dehydrate, and water for injection. The pH can be adjusted with citric acid or sodium hydroxide. The pH can be adjusted between 7 and 8.5 with citric acid and/or sodium hydroxide. A container (ampoule (11) or vial (10)) in a form (such as a suspension or solution) that is ready for injection can be a component of the kit.

One component of the medical kit can be alcohol swabs (pads), which is an antiseptic for preparation of the skin prior to injection. The alcohol swabs can be individually foil wrapped. The alcohol swabs can contain 70% isopropyl alcohol.

One component of the medical kit can be povidone iodine swab sticks (13) or a pad. Povidone-iodine is a broad-range anti-microbial and anti-fungal. Each swabstick or pad (13) can be saturated with a 10% PVP-I, USP solution equivalent to 1% available iodine. In one embodiment, a single package (12) containing three povidone iodine swabs (13) is a component of the kit. A medical practitioner can tear the package containing the swabs and take out the swabs to disinfect the injection site.

One component can be a Betadine® swab towelette or swabstick as an alternate format for cleaning the area prior to injection.

One component of the medical kit can be a pair of sterile gloves (14). The gloves (14) can be any suitable glove that is used by a medical practitioner, such as latex or nitrile gloves. In one embodiment, a pair of nitrile gloves are part of the kit. Nitrile gloves are a type of disposable glove made of Nitrile rubber, also known as Buna-N, Perbunan, or NBR. Nitrile gloves contain no latex. A medical practitioner can tear a package containing the gloves (14) and wear the gloves before performing an injection to help prevent contamination and increase compliance.

One component of the medical kit can be a bandage(s) (18, 19). The bandage can be a spot bandage (18), or a bandage in the shape of a strip (19) like a Band-Aid®, or other suitable bandage. The bandage (17) can be in a package to help prevent infection. A medical practitioner can tear the package (17) and use the bandage (18, 19) on a patient alter an injection is performed. Before a bandage (18, 19) is applied, the injection site can be disinfected with the iodine swab.

One component can be a small foil package of triple antibiotic ointment to be opened and help prevent infection. Active Ingredients of the antibiotic ointment can include Bacitracin, Neomycin, and Polymyxin B.

One component of the medical kit can be gauze (16, 20). Gauze (16, 20) is typically a thin, translucent fabric with a loose open weave. The gauze (16, 20) can be used to clean the injection site and/or secured to the injection site with the bandage (18, 19). A sufficient number of ply of gauze (20) can be provided with the kit, such in about two to about twenty ply, such as about 10 ply. The gauze (16, 20) can be any suitable size such as 2×2 to 6×6 size gauze, such as a 4×4 (in inches) size gauze.

One component can be a mask (21) to help prevent the spread of disease between the patient and the practitioner.

One component of the kit can be a sterile drape with a hole or aperture. The sterile drape is ideal for when the site around an injection need to be covered such as for an epidural injection. Kit #7 below can be used for an epidural injection.

One component of kit can be a sterile container of water (10 mL). One component of the kit can be a container of normal saline (10 mL) (preservative free). The water and saline can be used to flush the area before an injection.

One component of the kit can be an Ammonia inhalant ampule. Ammonia Inhalants (smelling salts) can be used for nasal inhalation in the prevention or treatment of fainting. In case of fainting (or initial symptoms), the ampoule package can be crushed and put under the nose of a patient. The ammonia ampule can contain 0.33 mL, with 35% alcohol and 15% ammonia.

One component of the kit can be a CSR (Central Supply Room) wrap. The CSR wrap can be put on a tray or a surface before placement of the other components of the kit. The CSR wrap keeps a clean environment.

The kit can comprise a holder such as a box (1) in which the above mentioned components are placed. The box (1) can be a rectangular cube which has a peel off lid (2) on top for opening the box.

The kit can include a tray (3). The tray (3) is put inside the box on bottom of the box (I) opposite of the lid (2) of the box. The tray (3) can have a plurality of cavities (4, 5, 6, 7, 8) for placing the components of the kit. In one embodiment, the tray has 5 cavities, with 4 cavities (5, 6, 7, 8) of different or same size in parallel fashion to each other, and a fifth cavity (4) abutting all the four cavities (5, 6, 7, 8) and having a area that is substantially same or bigger than combined area of the four cavities. The four cavities (5, 6, 7, 8) can be rectangular in shape and placed next to each other along their long side and abut the large cavity along the short side of the rectangle. In one embodiment, three of the four cavities (5, 6, 7) are the same size and the fourth cavity (8) is of smaller size.

The large cavity (4) allows for placement of a package of swabs, gauze, gloves, and bandage. The smaller cavities (5, 6, 7, 8) allow for placement of the active ingredients separately in each container. A medical practitioner can easily identify and locate ampoule (11) or vial (10) for each injection. The ampoule (11) or vial (10) can be placed inside its own box (9).

The following Table represents specific examples of components of the kits:

| Kit #1 | Kit #2 | Kit #3 | Kit #4 | Kit #5 | Kit #6 |
|---|---|---|---|---|---|
| Bupivacaine | Bupivacaine | Bupivacaine | Bupivacaine | Bupivacaine | |
| hydrochloride | hydrochloride | hydrochloride | hydrochloride | hydrochloride | |
| USP 0.5% | USP 0.5% | (10 ml) × 1 | (10 ml) × 1 | (10 ml) × 1 | |
| (10 ml) × 1 | (10 ml) × 1 | USP 0.5% | USP 0.5% | USP 0.5% | |
| (Marcaine ®) | (Marcaine ®) | (Marcaine ®) | (Marcaine ®) | (Marcaine ®) | |
| | Lidocaine | Lidocaine | Lidocaine | Lidocaine | Lidocaine |
| | HCl, USP | HCl, USP | HCl, USP | HCl, USP | HCl, USP |
| | 2% | 2% | 2% | 2% | 2% |
| | (20 mg/ml) | (20 mg/ml) | (20 mg/ml) | (20 mg/ml) | (20 mg/ml) |
| | (2 ml) × 1 | (2 ml) × 1 | (2 ml) × 2 | (2 ml) × 2 | (2 ml) × 2 |
| Triamcinolone | | Triamcinolone | Triamcinolone | Triamcinolone | Triamcinolone |
| acetonide | | acetonide | acetonide | acetonide | acetonide |
| 40 mg | | 40 mg | 40 mg | 40 mg | 40 mg |
| (1 ml) × 2 | | (1 ml) × 2 | (1 ml) × 3 | (1 ml) × 2 | (1 ml) × 1 |
| Kenalog-40 ® | | Kenalog-40 ® | Kenalog-40 ® | Kenalog-40 ® | Kenalog-40 ® |
| Povidone- | Povidone- | Povidone- | Povidone- | Povidone- | Povidone- |
| Iodine | Iodine | Iodine | Iodine | Iodine | Iodine |
| Swabsticks (3 | Swabsticks (3 | Swabsticks (3 | Swabsticks (3 | Swabsticks (3 | Swabsticks (3 |
| swabs) × 1 | swabs) × 1 | swabs) × 1 | swabs) × 1 | swabs) × 1 | swabs) × 1 |
| Pair of sterile | Pair of sterile | Pair of sterile | Pair of sterile | Pair of sterile | Pair of sterile |
| powder free | nitrile gloves | nitrile gloves | nitrile gloves | nitrile gloves | nitrile gloves |
| nitrile gloves | (size 7.5) × 1 | (size 7.5) × 1 | (size 7.5) × 1 | (size 7.5) × 1 | (size 7.5) × 1 |
| (size 7.5) × 1 | | | | | |
| Spot | Spot | Adhesive | Adhesive | Adhesive | Adhesive |
| Adhesive | Adhesive | Bandage × 1 | Bandage × 1 | Bandage × 1 | Bandage × 1 |
| Bandage × 1 | Bandage × 1 | | | | |
| 4 × 4 Non | 4 × 4 Non | 4 × 4 Non | 4 × 4 Non | 4 × 4 Non | 4 × 4 Non |
| Sterile Gauze | Sterile Gauze | Sterile Gauze | Sterile Gauze | Sterile Gauze | Sterile Gauze |
| at least 10 ply | at least 10 ply | at least 10 ply | at least 10 ply | at least 10 ply | at least 10 ply |
| Kit #7 | Kit #8 | Kit #9 | Kit #10 | Kit #10A | |
| Methylprednisolone | Methylprednisolone | Methylprednisolone | | | |
| acetate Injection | acetate | acetate 40 mg/ml | | | |
| 40 mg/mL (1-5 mL) | 40 mg/ml (5 mL) × 1 | (1 mL vial) × 1 | | | |
| (Depo-Medrol ®) | (Depo-Medrol ®) | (Depo-Medrol ®) | | | |
| | | | Triamcinolone | Triamcinolone | |
| | | | acetonide | acetonide | |
| | | | (1 ml) × 2 40 mg | (1 ml) × 1 | |
| | | | (Kenalog-40 ®) | (Kenalog-40 ®) | |
| Lidocaine HCl USP | Lidocaine HCl USP | Lidocaine HCl USP | Lidocaine HCl USP | Lidocaine HCl USP | |
| 1% (10-20 mL) × 1 | 1% (2 mL) × 2 | 1% (10 mg/ml) | 1% (10 mL) | 1% (2 mL) | |
| | | (2 mL ampule) × 2 | (10 mg/ml) × 1 | (10 mg/ml) × 1 | |
| Marcaine HCl 0.5% | | | | | |
| (10 mL) × 1 | | | | | |
| (Bupivacaine | | | | | |
| hydrochloride). | | | | | |
| Alcohol Swab × 1 | Alcohol swabs × 6 | Alcohol swabs × 6 | | | |
| | | | Ammonia | Ammonia | |
| | | | inhalant | inhalant | |
| | | | ampule × 1 | ampule × 1 | |
| | | | (35% | (35% | |
| | | | alcohol/15% | alcohol/15% | |
| | | | ammonia) | ammonia) | |
| Povidone-Iodine | | | Povidone- | Povidone- | |
| Swabsticks (3 | | | iodine prep | iodine prep | |
| swabs) × 1 | | | pads × 1 | pads × 1 | |
| | Gauze | Gauze | | | |
| Sterile Nitrile | Sterile Nitrile | Sterile Nitrile | Sterile Latex | Sterile Latex | |
| Gloves (size 7.5) | Gloves (size 7.5) | Gloves (size 7.5) | Gloves (size 7) | Gloves (size 7) | |
| Bandage | Bandage | Bandage | | | |
| Sterile drape (w/ | | | CSR Wrap | CSR Wrap | |
| hole) × 1 | | | | | |

| Kit #11 | Kit #12 | | |
|---|---|---|---|
| 5 mL Lidocaine HCl | 5 mL Lidocaine HCl | | |
| USP 1% (pf) × 1 | USP 1% (pf) × 1 | | |
| (10 mg/ml) (5 mL) | (10 mg/ml) (5 mL) | | |
| 10 mL bupivacaine | 10 mL bupivacaine | | |
| hydrochloride | hydrochloride | | |
| 0.25% × 1 (10 mL) | 0.25% × 1 (10 mL) | | |
| (Marcaine ®) | (Marcaine ®) | | |
| 1 mL | 1 mL | | |
| Methylprednisolone | Methylprednisolone | | |
| acetate | acetate | | |
| Depo-Medrol ® | Depo-Medrol ® | | |
| 40 mg/mL × 1 | 80 mg/mL × 1 | | |
| 10 mL Sterile | 10 mL Sterile | | |
| Water × 1 | Water × 1 | | |
| 10 mL Normal | 10 mL Normal | | |
| Saline (pf) × 1 | Saline (pf) × 1 | | |
| Sterile drape with | Sterile drape with | | |
| hole × 1 | hole × 1 | | |
| Povidone-Iodine | Povidone-Iodine | | |
| Swabsticks (3 | Swabsticks (3 | | |
| swabs) × 1 | swabs) × 1 | | |
| Nitrile Powder Free | Nitrile Powder Free | | |
| Sterile Gloves (size | Sterile Gloves (size | | |
| 7.5) × 1 | 7.5) × 1 | | |
| Alcohol Swab × 1 | Alcohol Swab × 1 | | |
| Kit #13 | Kit #14 | Kit #15 | Kit #19 |
| 1 mL Ketorolac | 1 mL Ketorolac | | Ketorolac |
| Tromethamine | Tromethamine | | Tromethamine |
| 30 mg/ml × 1 | 30 mg/ml × 1 | | Inj., USP |
| Toradol ® | Toradol ® | | 30 mg/mL × 1 |
| | | | Toradol |
| 2 mL Lidocaine | 2 mL Lidocaine | 2 mL Lidocaine | Lidocaine HCl |
| HCl USP 1% × 1 | HCl USP 1% × 1 | HCl USP 2% × 1 | Injection USP 1% |
| 10 mg/ml | | | (2 mL) × 1 |
| | | | 10 mg/ml |
| | 10 mL | 10 mL | Marcaine ™ 0.25% |
| | Marcaine ™ | Marcaine ™ | (10 mL) × 1 |
| | 0.25% × 1 | 0.25% × 1 | Bupivacaine |
| | Bupivacaine | Bupivacaine | hydrochloride |
| | hydrochloride | hydrochloride | |
| Povidone-Iodine | Povidone-Iodine | Povidone-Iodine | Povidone-Iodine |
| Swabsticks (3 | Swabsticks (3 | Swabsticks (3 | Swabsticks (3 |
| swabs) × 1 | swabs) × 1 | swabs) × 1 | swabs) × 1 |
| Nitrile Powder | Nitrile Powder | Nitrile Powder | Pair Nitrile |
| Free Sterile | Free Sterile | Free Sterile | Powder Free |
| Gloves | Gloves | Gloves | Sterile Glove |
| (size 7.5) × 1 | (size 7.5) × 1 | (size 7.5) × 1 | (Non-Latex)* |
| | | | (Size 7.5) × 1 |
| Adhesive | Bandage × 1 | | Adhesive |
| Bandage × 1 | | | Bandage × 1 |
| 4 × 4 Non-sterile | 4 × 4 Non-sterile | 4 × 4 Non-sterile | 4 × 4 Non-sterile |
| Gauze | Gauze | Gauze | Gauze |
| | | Spot Adhesive | |
| | | Bandage × 1 | |
| Kit #16 | Kit #17 | Kit #20 | Kit #21 |
| Betamethasone | Dexamethasone | Betamethasone | Dexamethasone |
| Sodium | Sodium | Sodium | Sodium |
| Phosphate and | Phosphate Inj., | Phosphate and | Phosphate Inj., |
| Betamethasone | UPS 10 mg/mL | Betamethasone | UPS 10 mg/mL |
| Acetate Injection | (1 mL) × 1 | acetate | (1mL) × 1 |
| Suspension, USP | | 30 mg/5 mL | |
| 30 mg/5 mL | | (6 mg/mL) | |
| (6 mg/mL) | | (5 mL) × 1 | |
| 5 mL × 1 | | | |
| Lidocaine HCl | Lidocaine HCl | | |
| Injection, USP | Injection, USP | | |
| 1% (10 mg/ml) | 1% (10 mg/ml) | | |
| (10 mL) × 1 | (2 mL) × 1 | | |
| | | Marcaine ™ | Marcaine ™ |
| | | 0.25% (10 mL) | 0.25% (2.5 mg) |
| | | (2.5 mg) × 1 | (10 mL) × 1 |
| | | Bupivacaine | Bupivacaine |
| | | hydrochloride | hydrochloride |
| Povidone-Iodine | Povidone-Iodine | Povidone-Iodine | Povidone-Iodine |
| Swabsticks (1 × 3 | Swabsticks (1 × 3 | Swabsticks (3 | Swabsticks (3 |
| Swabs) | Swabs) | Swabs) × 1 | Swabs) × 1 |
| Pair Nitrile | Pair Nitrile | Pair Nitrile | Pair Nitrile |
| Powder Free | Powder Free | Powder Free | Powder Free |
| Sterile Glove | Sterile Glove | Sterile Glove | Sterile Glove |
| (Non-Latex) | (Non-Latex) | (Non-Latex) | (Non-Latex) |
| (Size 7.5) | (Size 7.5) | (Size 7.5) | (Size 7.5) |
| Non Sterile 4 × 4 | Non Sterile 4 × 4 | Non Sterile 4 × 4 | Non Sterile 4 × 4 |
| Gauze | Gauze | Gauze | Gauze |

| Kit #22 | Kit #23 | |
|---|---|---|
| Methylprednisolone acetate | Methylprednisolone acetate | |
| Depo-Medrol ® 40 mg/ml | Depo-Medrol ® 80 mg/ml | |
| (1 × 1 mL) | (1 × 1 mL) | |
| Bupivacaine hydrochloride | Bupivacaine hydrochloride | |
| Marcaine ™ 0.25% (10 mL) | Marcaine ™ 0.25% (10 mL) | |
| Lidocaine HCl Injection, USP | Lidocaine HCl Injection, USP | |
| 1% (5 mL) | 1% (5 mL) | |
| Sterile Water for Inj., USP | Sterile Water for Inj., USP | |
| (10 mL × 1) | (1 × 10 mL) | |
| 0.9% Sodium Chloride Inj., | 0.9% Sodium Chloride Inj., | |
| USP (10 mL × 1) | USP (1 × 10 mL) | |
| Povidone-Iodine Swabsticks | Povidone-Iodine Swabsticks | |
| (1 × 3 Swabs) | (1 × 3 Swabs) | |
| Isopropyl Alcohol 70% Prep | Isopropyl Alcohol 70% Prep | |
| Pads (5) | Pads (5) | |
| Pair Nitrile Powder Free | Pair Nitrile Powder Free | |
| Sterile Glove (Non-Latex)* | Sterile Glove (Non-Latex)* | |
| (Size 7.5) | (Size 7.5) | |
| Drape with Fenestration | Drape with Fenestration | |
| Kit #24 | Kit #25 | Kit #26 |
| Marcaine ™ 0.25% | Dexamethasone Sodium | Methylprednisolone acetate |
| (10 mL) × 1 | Phosphate Inj., UPS 10 mg/mL | USP 40 mg/mL multiple dose |
| Bupivacaine hydrochloride | (1 mL) × 1 | vial (5 mL) × 1 |
| Lidocaine HCl Injection, | Lidocaine HCl Injection, | |
| USP 1% (2 mL) × 1 | USP 1% (2 mL) × 1 | |
| (10 mg/ml) | (10 mg/ml) | |
| | Marcaine ™ 0.25% (10 mL) | Marcaine ™ 0.5% (10 mL) |
| | Bupivacaine hydrochloride × 1 | Single dose vial |
| | | Bupivacaine hydrochloride × 1 |
| Povidone-Iodine Swabsticks | Povidone-Iodine Swabsticks | Povidone-Iodine Prep pad |
| (3 Swabs) × 1 (Dynarex ™) | (3 Swabs) × 1 | |
| Pair Nitrile Powder Free | Pair Nitrile Powder Free | Pair of sterile latex gloves- |
| Sterile Glove (Non-Latex)* | Sterile Glove (Non-Latex)* | size 7 |
| (Size 7.5) × 1 | (Size 7.5) × 1 | |
| Spot Adhesive Bandage × 1 | | |
| Non Sterile 4 × 4 Gauze | Non Sterile 4 × 4 Gauze | |
| | | 1 CSR wrap |

| Kit #27 | Kit #28 |
|---|---|
| Bupivacaine hydrochloride | Dexamethasone Sodium |
| Marcaine ™ 0.5% single dose | Phosphate Inj., UPS 10 mg/mL |
| vial (10 mL) × 1 | (1 mL) × 2 |
| Lidocaine HCl | |
| USP 2% single dose ampule | |
| (20 mg/ml) | |
| (2 mL) × 1 | |
| Triamcinolone acetonide | |
| (1 ml) × 2 40 mg | |
| (Kenalog-40 ®) | |
| Povidone-iodine swabsticks (3 | Povidone-iodine swabsticks (3 |
| swabs) × 1 | swabs) × 1 |
| 1 pair nitrile powder free sterile | 1 pair nitrile powder free sterile |
| gloves (size 7.5) | gloves (size 7.5) |
| 1 adhesive bandage | |
| Non-sterile 4 × 4 gauze | Non-sterile 4 × 4 gauze |

Kit No. 1 can have triamcinolone acetonide, bupivacaine hydrochloride in separate cavities, and the gloves, swabs, gauze, and bandage placed in the large cavity (4). The four cavities can include bupivacaine hydrochloride (cavity 1), triamcinolone acetonide (cavity 2), triamcinolone acetonide (Cavity 3), and an empty fourth cavity.

Kit No. 2 can have Bupivacaine hydrochloride, gauze, and lidocaine HCl in separate cavities, and the gloves, swabs, gauze, and bandage placed in the large cavity (4). The four cavities can include bupivacaine hydrochloride (cavity 1), empty cavity 2, gauze (Cavity 3), and lidocaine HCl (cavity 4).

Kit No. 3 can have bupivacaine hydrochloride, lidocaine, triamcinolone acetonide, and triamcinolone acetonide in separate cavities, and the gloves, swabs, gauze, and bandage placed in the large cavity (4). The four cavities can include bupivacaine hydrochloride (cavity 1), triamcinolone acetonide (cavity 2), triamcinolone acetonide (Cavity 2), and lidocaine (cavity 4).

Kit. No. 4 can have bupivacaine hydrochloride, triamcinolone acetonide, triamcinolone acetonide, lidocaine in separate cavities, and the gloves, swabs, bandage, gauze, and another container of triamcinolone acetonide placed in the large cavity (4). The four cavities can include bupivacaine hydrochloride (cavity 1), triamcinolone acetonide (Cavity 2), triamcinolone acetonide (Cavity 3), and lidocaine (Cavity 3), and lidocaine (Cavity 4) (and an additional container of triamcinolone acetonide placed in the large cavity).

Kit. No. 5 can have bupivacaine hydrochloride, triamcinolone acetonide, triamcinolone acetonide, lidocaine in separate cavities, and the gloves, swabs, bandage, and gauze placed in the large cavity (4). The four cavities can include bupivacaine hydrochloride (cavity 1), triamcinolone acetonide (Cavity 2), triamcinolone acetonide (Cavity 3), lidocaine (Cavity 3), and lidocaine (Cavity 4).

Kit. No. 6 can have triamcinolone acetonide, lidocaine, and gauze in separate cavities, and the gloves, swabs, bandage placed in the large cavity (4). The four cavities can include triamcinolone acetonide (cavity 1), gauze (Cavity 2), triamcinolone acetonide, two containers of lidocaine (Cavity 3), and an empty fourth cavity.

The components of Kits Nos. 7 to 23 can be placed in the tray in similar manner.

The kit can contain an instruction sheet. The instruction sheet can identify one or more components of the kit, provide instructions as to how to use each component, and optionally suggest an order of use for the components of the kit. The instruction sheet can provide instructions as to where and how to inject each of the active ingredients.

) Instead of an instruction sheet, each component of the kit can be described on the box (1) of the kit, for example on opposite side of the lid (2) of the box. Each component can have its own separate instruction sheet provided by its own manufacturer.

For the above embodiments, cavity 1 refers to (5), cavity 2 refers to (6), cavity 3 refers to (7), and cavity 4 refers to (8).

Also provided is a method of treating or reducing pain or inflammation by using any of the above kits.

What is claimed is:

1. A kit comprising:
a) a holder;
b) one or more active pharmaceutical ingredients (API), with each API placed in a separate container in a form that is ready for injection, the API placed in the holder of the kit selected from the group consisting of:
a. lidocaine hydrochloride and triamcinolone acetonide with or without a separate container of ammonia;
b. bupivacaine hydrochloride (HCl) and lidocaine hydrochloride (HCl), and optionally one or more of triamcinolone acetonide, methylprednisolone acetate, or Dexamethasone Sodium;
c. bupivacaine hydrochloride (HCl) and one or more of triamcinolone acetonide or methylprednisolone acetate;
d. Methylprednnisolone acetate and lidocaine hydrochloride (HCl), and optionally bupivacaine hydrochloride (HCl);
e. Methylprednisolone acetate and lidocaine hydrochloride (HCl) and bupivacaine hydrochloride (HCl);
f. lidocaine hydrochloride, triamcinolone acetonide, and ammonia;
g. Bupivacaine hydrochloride or lidocaine HCl, and Betamethasone Sodium Phosphate and Betamethasone acetate;
h. Bupivacaine hydrochloride or lidocaine HCl, and Dexamethasone Sodium phosphate;
i. ketorolac tromethamine, lidocaine HCl, and optionally Bupivacaine hydrochloride; and
j. one or more of dexamethasone Sodium.

2. The kit of claim 1, wherein the holder is a box.

3. The kit of claim 2, further comprising a tray placed inside the box.

4. The kit of claim 1, wherein the kit further comprises three or more of the following: antibiotic, gloves, mask, bandage, gauze, alcohol swabs, sterile water, saline water, a sterile drape, CSR wrap, povidone-iodine pads or swabsticks, and instruction sheet.

5. The kit of claim 1, wherein the kit comprises lidocaine hydrochloride and triamcinolone acetonide with or without a separate container of ammonia.

6. The kit of claim 1, wherein the kit comprises bupivacaine hydrochloride (HCl) and lidocaine hydrochloride (HCl), and optionally one or more of triamcinolone acetonide, methylprednisolone acetate, or dexamethasone Sodium.

7. The kit of claim 1, wherein the kit comprises bupivacaine hydrochloride (HCl) and one or more of triamcinolone acetonide or methylprednisolone acetate.

8. The kit of claim 1, wherein the kit comprises methylprednisolone acetate and lidocaine hydrochloride (HCl), and optionally bupivacaine hydrochloride (HCl).

9. The kit of claim 1, wherein the kit comprises methylprednisolone acetate and lidocaine hydrochloride (HCl), and bupivacaine hydrochloride (HCl).

10. The kit of claim 1, wherein the kit comprises lidocaine hydrochloride, triamcinolone acetonide, and ammonia.

11. The kit of claim 1, wherein the kit comprises bupivacaine hydrochloride or lidocaine HCl, and betamethasone sodium phosphate and betamethasone acetate.

12. The kit of claim 1, wherein the kit comprises bupivacaine hydrochloride or lidocaine HCl, and dexamethasone sodium phosphate.

13. The kit of claim 1, wherein the kit comprises ketorolac tromethamine, lidocaine HCl, and optionally bupivacaine hydrochloride.

14. The kit of claim 1, wherein the kit comprises one or more of dexamethasone Sodium.

15. The kit of claim 1, wherein the holder is a box and the kit comprises:
a) One container of bupivacaine hydrochloride;
b) Two containers of triamcinolone acetonide;
c) One or more packages of povidone-iodine swabsticks;
d) gloves;
e) bandage; and
f) gauze.

16. The kit of claim 1, wherein the holder is a box and the kit comprises:
a) One container of bupivacaine hydrochloride;
b) One container of lidocaine HCl;
c) One or more povidone-iodine swabsticks;
d) gloves;
e) bandage; and
g) gauze.

17. The kit of claim 1, wherein the holder is a box and the kit comprises:
a) One container of bupivacaine hydrochloride;
b) One container of lidocaine HCl;
c) Two containers of triamcinolone acetonide;
d) One or more povidone-iodine swabsticks;
e) gloves;
f) bandage; and
g) gauze.

18. The kit of claim 1, wherein the holder is a box and the kit comprises:
a) One container of bupivacaine hydrochloride;
b) Two containers of lidocaine HCl;
c) Three containers of triamcinolone acetonide;
d) One or more povidone-iodine swabsticks;
e) gloves;
f) bandage; and
g) gauze.

19. The kit of claim 1, wherein the holder is a box and the kit comprises:
a) One container of bupivacaine hydrochloride;
b) Two containers of lidocaine HCl;
c) Two containers of triamcinolone acetonide;
d) One or more povidone-iodine swabsticks;
C) gloves;
f) bandage; and
g) gauze.

20. The kit of claim 1, wherein the holder is a box and the kit comprises:
a) Two containers of lidocaine HCl;
b) One container of triamcinolone acetonide;
c) One or more povidone-iodine swabsticks;
d) gloves;
e) bandage; and
f) gauze.

21. The kit of claim 1, wherein the holder is a box and the kit comprises:
a) one container of methylprednisolone acetate;
b) two containers of lidocaine HCl;
c) one or more of alcohol swabs;
d) gauze;
e) gloves; and
f) bandage.

22. The kit of claim 1, wherein the holder is a box and the kit comprises:
a) two containers of triamcinolone acetonide;
b) one container of lidocaine HCl;
c) one or more containers of ammonia inhalant;
d) one or more of povidone-iodine prep pads;
e) gloves; and
f) CSR wrap.

23. The kit of claim 1, wherein the holder is a box and the kit comprises:
a) one container of triamcinolone acetonide;
b) one container of lidocaine HCl;
c) one or more container of ammonia inhalant ampule;
d) one or more of povidone-iodine pads;
e) a package of gloves; and
f) CSR wrap.

24. The kit of claim 1, wherein the holder is a box and the kit comprises:
a) one container of ketorolac tromethamine;
b) one container of lidocaine HCl;
c) one or more of povidone-iodine swabsticks;
d) gloves;
e) bandage; and
f) gauze.

25. The kit of claim 1, wherein the holder is a box and the kit comprises:
a) one container of ketorolac tromethamine;
b) one container of lidocaine HCl;
c) one container of bupivacaine hydrochloride;
d) one or more of povidone-iodine swabsticks;
e) gloves;
f) bandage; and
g) gauze.

26. The kit of claim 1, wherein the holder is a box and the kit comprises:
a) one container of lidocaine HCl;
b) one container of bupivacaine hydrochloride;
c) one or more of povidone-iodine swabsticks;
d) gloves;
e) gauze; and
f) bandage.

27. The kit of claim 1, wherein the holder is a box and the kit comprises:
a) one container of betamethasone;
b) one container of lidocaine HCl;
c) one or more of povidone-iodine swabsticks;
d) gloves; and
e) gauze.

28. The kit of claim 27, wherein the betamethasone is in form of betamethasone Sodium Phosphate and betamethasone Acetate.

29. The kit of claim 1, wherein the holder is a box and the kit comprises:
a) one container of dexamethasone sodium phosphate;
b) one container of lidocaine HCl;
c) one or more povidone-iodine swabsticks;
d) gloves; and
e) gauze.

30. The kit of claim 1, wherein the holder is a box and the kit comprises:
a) one container of ketorolac tromethamine;
b) one container of lidocaine HCl;
c) one container of bupivacaine hydrochloride;
d) one or more povidone-iodine Swabsticks;
e) gloves;
t) bandage; and
g) gauze.

31. The kit of claim 1, wherein the holder is a box and the kit comprises:
a) one container of betamethasone;
b) one or more containers of bupivacaine hydrochloride;
c) one or more povidone-iodine swabsticks;
d) gloves; and
e) gauze.

32. The kit of claim 31, wherein the betamethasone is in form of betamethasone Sodium phosphate and betamethasone acetate.

33. The kit of claim 1, wherein the holder is a box and the kit comprises:
a) one container of dexamethasone sodium phosphate;
b) one container of bupivacaine hydrochloride;
c) one or more povidone-iodine swabsticks;
d) gloves; and
e) gauze.

34. The kit of claim 1, wherein the holder is a box and the kit comprises:
a) one container of methylprednisolone acetate;
b) one container of bupivacaine hydrochloride;
c) one container of lidocaine HCl;
d) one container of saline;
e) one container of water;
f) one or more povidone-iodine swabsticks;
g) one or more alcohol pads,
h) gloves; and
i) drape.

35. The kit of claim 34, wherein the methylprednisolone acetate is present in 40 mg or 80 mg.

36. The kit of claim 1, wherein the holder is a box and the kit comprises:
a) One container of bupivacaine hydrochloride;
b) One container of lidocaine HCl;
c) One or more povidone-iodine swabsticks;
d) gloves;
c) bandage; and
f) gauze.

37. The kit of claim 1, wherein the holder is a box and the kit comprises:
a) One container of dexamethasone sodium phosphate;
b) One container of lidocaine HCl;
c) One container of bupivacaine hydrochloride;
d) one or more povidone-iodine swabsticks;
e) gloves; and
f) gauze.

38. The kit of claim 1, wherein the holder is a box and the kit comprises:
a) One container of methylprednisolone acetate;
b) One container of bupivacaine hydrochloride;
c) one or more povidone-iodine pads;
d) gloves; and
e) CSR wrap.

39. The kit of claim 1, wherein the holder is a box and the kit comprises:
a) one container of bupivacaine hydrochloride;
b) one container of lidocaine HCl;
c) two containers of triamcinolone acetonide;
d) one or more povidone-iodine swabsticks;
e) gloves;
t) bandage; and
g) gauze.

40. The kit of claim 1, wherein the holder is a box and the kit comprises:
a) Two containers of dexamethasone sodium phosphate;
b) One or more povidone-iodine swabsticks;
c) gloves; and
d) gauze.
